SEQUENCE LISTING (4 sequences)

SEQ ID NO: 1 - DNA, 57 bp, Artificial Sequence
  Synthetic construct
ccggcaacaagatgaagagcaccaactcgagttggtgctcttcatcttgttgttttt

SEQ ID NO: 2 - DNA, 57 bp, Artificial Sequence
  Synthetic construct
ccggccctgaaatcacagtctccaactcgagttggagactgtgatttcagggttttt

SEQ ID NO: 3 - DNA, 57 bp, Artificial Sequence
  Synthetic construct
ccggcgactatcagactattatcaactcgagttgataatagtctgatagtcgttttt

SEQ ID NO: 4 - DNA, 1581 bp, Homo sapiens
atggacgtaagattttatccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctcccagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcagacatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgccagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttga

(19) United States
(12) Patent Application Publication
(10) Pub. No.: US 2016/0002626 A1
(43) Pub. Date: Jan. 7, 2016

(54) TOX INHIBITION FOR THE TREATMENT OF CANCER

(71) Applicants: Youwen ZHOU, (US); Yuanshen HUANG, (US); Yang WANG, (US); Ming-wan SU, (US); Xiaoyan JIANG, (US)

(72) Inventors: Youwen Zhou, Vancouver (CA); Yuanshen Huang, Vancouver (CA); Yang Wang, Beijing (CN); Ming-wan Su, Vancouver (CA); Xiaoyan Jiang, Vancouver (CA)

(21) Appl. No.: 14/647,699

(22) PCT Filed: Nov. 28, 2013
(86) PCT No.: PCT/CA2013/000998

§ 371 Date: May 27, 2015

Related U.S. Application Data
(60) Provisional application No. 61/730,666, filed on Nov. 28, 2012.

(51) Int. Cl. C12N 15/113; A61K 31/7088; A61K 39/395; A61K 47/48; A61K 9/00; A61K 45/06; A61K 31/436; C07K 16/18

(52) U.S. Cl. CPC C12N 15/113; A61K 31/436; A61K 31/7088; C07K 16/18; A61K 47/4833; A61K 9/0014; A61K 45/06; A61K 39/39558; C12N 2310/3233; C12N 2310/14; C12N 2310/122; C12N 2310/531; C12N 2320/31; C07K 2317/76

(57) ABSTRACT

Described are methods for reducing the proliferation cancer cells by modulating the expression or activity of TOX such as by use of a TOX inhibitor. Inhibiting TOX expression with antisense nucleic acids is shown to reduce the proliferation of malignant T cells. Also described are methods for the treatment of cancer in a subject in need thereof comprising administering to the subject a TOX inhibitor. Optionally, the cancer is a T cell malignancy such as Cutaneous T cell Lymphoma (CTCL).

### RELATED APPLICATIONS

This application claims priority to U.S. Provisional Patent Application No. 61/730,666 filed Nov. 28, 2012, the contents of which are incorporated by reference herein in their entirety.

### FIELD OF THE DISCLOSURE

The disclosure relates to methods for the treatment of cancer and more specifically to therapeutic methods and associated uses for treating T cell malignancies by inhibiting TOX.

### BACKGROUND OF THE DISCLOSURE

There are a group of T cell derived malignancies affecting humans, including cutaneous T cell lymphomas (CTCL), peripheral T cell lymphomas, T cell leukemias, and their histological and clinical variants. Although the combined overall incidence of CTCL is low, at less than 10 per million population per year, they are often difficult to differentiate from far more common disease conditions, such as chronic dermatitis (approximately 10% of the general population), cutaneous reaction to drugs (1-5% of population), psoriasis (1.5% of population) and pityriasis rubra pilaris (approximately 0.1% of population), especially at an early stage of disease.

Jain et al. (2012) describe a number of therapeutic agents for the treatment of CTCL, however many cases of T cell malignancy are unresponsive to available treatments or difficult to control.

There is a need for new treatments and therapeutics for the treatment of T cell malignancy.

### SUMMARY OF THE DISCLOSURE

The inventors have determined that modulation of TOX is useful for reducing the proliferation of cancer cells. As shown in Example 1, TOX gene knock-down resulted in a growth disadvantage to malignant T cells (Hut78) relative to controls in a cell viability assay. Knock-down of the TOX gene was also shown to reduce colony size and number relative to controls in a colony forming assay. As shown in Example 2, knock-down of the TOX gene in another CTCL cell line (HH) also resulted in a growth disadvantage and reduced colony size and number relative to control cells. The inventors have also determined that TOX knock-down results in G0/G1 and G1/S phase arrest in cancer cells and that inhibition of TOX sensitizes malignant T cells to FasL induced apoptosis. TOX inhibition has also been demonstrated to result in a significant increase in spontaneous apoptosis in CTCL cancer cells.

Accordingly, in one aspect there is provided a method of reducing the proliferation of cancer cells comprising modulating the expression or activity of TOX in the cancer cells. In one embodiment, there is provided a method of reducing the proliferation of cancer cells comprising contacting the cells with a TOX inhibitor. In one embodiment, there is provided the use of a TOX inhibitor for reducing the proliferation of cancer cells.

In some embodiments, the cancer cells are malignant T cells, such as Cutaneous T-cell Lymphoma (CTCL) cells, peripheral T-cell lymphoma cells or leukemic T cells. Optionally, the cells may be in vivo or in vitro.

In one aspect there is provided a method for the treatment of cancer in a subject in need thereof, comprising modulating the transcription, translation or activity of TOX. In one embodiment, there is provided a method of treating cancer in a subject in need thereof, comprising administering to the subject a TOX inhibitor. Also provided is the use of a TOX inhibitor for the treatment of cancer. Also provided is a TOX inhibitor for use in the treatment of cancer. Also provided is the use of a TOX inhibitor for the manufacture of a medicament for the treatment of cancer. In one embodiment, the methods and uses described herein include the administration or use of one or more additional chemotherapeutic agents, such as an agent that induces apoptosis. In one embodiment, the methods described herein include co-administration or use of a TOX inhibitor and one or more chemotherapeutic agents selected from methotrexate, retinoids, or histone deacytylase modifiers. In one embodiment, the retinoid is selected from Acitretin (Soriatane®) and bexarotene. In one embodiment the histone deacytylase modifier is selected from vorinostat and romidepsin.

In some embodiments, the cancer is a T cell malignancy, such as Cutaneous T-cell Lymphoma (CTCL), peripheral T-cell lymphoma or T cell leukemia. In one embodiment, the cancer is Mycosis Fungoides (MF) or Sezary Syndrome.

In one aspect, the methods described herein for reducing cell proliferation or for the treatment of cancer involve the use of a TOX inhibitor. In one embodiment, the TOX inhibitor prevents or reduces the expression of TOX or reduces the activity of the TOX protein. For example, in some embodiments, the TOX inhibitor prevents the transcription of TOX mRNA or translation of TOX protein. In one embodiment, the TOX inhibitor is a nucleic acid that binds to a nucleic acid encoding for all or part of TOX, such as a small hairpin RNA (shRNA), small interfering RNA (siRNA) or morpholino oligonucleotide. In one embodiment, the TOX inhibitor is a compound that binds to the TOX protein and inhibits the activity of TOX, such as a TOX antibody. Optionally, the TOX inhibitor may be generated in vivo in a subject such as by the use of gene therapy to express of one or more TOX inhibitors. In some embodiments, the TOX inhibitor is a regulator of up-stream molecular steps, such as transcription factors RUNX1, RUNX3 and their activity modulators in all forms. In one embodiment, the TOX inhibitor is an inhibitor of calcineurin activity, such as FK506 (also known as tacrolimus or fujimycin and available under the trade names Prograf™, Advagraf™ and Protopic™) pimecrolimus, or cyclosporine and related compounds. Optionally, calcineurin inhibitors may be used in systemic forms or in topical forms to modulate TOX expression in a subject to reduce cell proliferation or for the treatment of cancer.

In one embodiment, the TOX inhibitor is conjugated to another molecule that improves the delivery, safety or efficacy of the TOX inhibitor. In one embodiment, the TOX inhibitor, such as a siRNA that binds to TOX mRNA or a calcineurin inhibitor such as FK506, is conjugated to a cell-penetrating peptide. In one embodiment, the cell penetrating peptide is selected from TAT, Angiopep, penetratin, TP, rabies, virus glycoprotein (RVG), prion peptide, and SynB or other penetration enhancers known in the art. In one embodiment, the TOX inhibitor is conjugated to polyethylene glycol (PEG). In one embodiment, the TOX inhibitor is conjugated to another molecule that improves the transdermal delivery of the TOX inhibitor. In one embodiment, the TOX inhibitor is in a pharmaceutically acceptable formulation. For example, in one embodiment the TOX inhibitor is in a pharmaceutically acceptable formulation for the transdermal delivery of the TOX inhibitor.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1 shows the construct and inserts used for Lentivirus-mediated TOX gene knock-down. (A) Non-targeting control with insert sequence CCGGCAACAAGATGAAGAGCACCAACTCGAGTTGGTGCTCTT CATCTTGTTGTTTTT (SEQ ID NO: 1) (SHC002; available from Sigma Aldrich™) and TOX shRNA sequences TOX sh2 CCGGCCCTGAAATCACAGTCTCCAACTCGAGTTGGAGACTGTGATTTCG GGTTTTT (SEQ ID NO: 2) and TOX sh3 CCGGCGACTATCAGAC TATTATCAACTCGAGTTGATAATAGTCTGATAGTCGTTTTT (SEQ ID NO: 3) and the vector backbone structures are shown. Inserts were verified by Sanger sequencing. Helper-free virus was generating by transfecting HEK 293T packaging cells cultured in Dulbecco modified Eagle medium plus 10% fetal bovine serum (StemCell™ Technologies) using calcium-phosphate precipitation method. The virus-containing medium was harvested and filtered 24 to 48 hours later. Hut78 cells were resuspended at 1×10⁵ cells/mL in the virus-containing medium diluted 1:2 in RPMI-1640 containing 10% fetal bovine serum and protamine sulfate (5 mg/mL) for 24 hours. The cells were incubated at 37° C. for a further 48 hours. Puromycin (1 μg/ml) was then added to the culture media to select for virus-transduced population. In the following cell assays, both the vector-transduced control cells (Hut78-SHC002) and the parental untransduced cells (Hut78) served as controls. (B) TOX expression level was evaluated by Western blotting using specific anti-TOX antibody (Sigma Aldrich) 7 days post virus infection. Bulk populations transduced with TOX shRNA showed significant reduction of TOX expression, compared with control populations. FIG. 1B is representative of 4 independent transductions. Actin served as an internal reference.

FIG. 2 presents cell viability assays showing that Lentivius-mediated TOX gene transduction results in growth disadvantage to Hut78 cells. Hut78-SHC002 (solid line) cells display growth advantage to Hut78 cell (double solid line) over TOX shRNA cells (TOX knock-down, dotted line). On day 4, the viable cells of Hut78-SHC002 and Hut78 cells were significantly higher than those of TOX shRNA cells. Data were generated from lentivirus transduced bulk cell populations. All experiments were triplicated. P<0.005** by Mann-Whitney U test.

FIG. 3 shows a colony forming cell assay wherein knock-down of TOX gene results in a proliferation disadvantage to Hut78 cells. Hut78-SHC002 cells presented higher proliferation capacity to Hut78 cell over TOX shRNA cells. On day 11, the colony size (A) and number (B) of Hut78 TOX shRNA cells were significantly smaller than those of Hut78-SHC002 cells. Data were generated from lentivirus transduced bulk cell populations. All experiments were duplicated, and biological replicates were performed 3 times. P=0.0022***by Mann-Whitney U test. Magnification 20×.

FIG. 4 shows that TOX knock down results in G0/G1 and G1/S phase arrest in Hut78 cells. Hut78 cells and Hut78 cells transduced with control (SHC002) or TOX shRNA were incubated with Hoechst and Pyronin and analyzed by FACS analysis. Numbers denote the percentage of cells within the quadrant. (B) Cell-cycle profiles of Hut78 cells and Hut78 cells transduced with control and TOX shRNA. TOX shRNA transduced cells had more cells in the G0 phase and less cells in the S+G2M phase, compared with Hut78 cells and control vector transduced Hut78 cells. Data in the bar graphs are expressed as the mean±SD of 4 independent transductions and untransduced Hut78 cells. Numbers denote the percentage of cells within the quadrant.

FIG. 5 shows that TOX knock-down sensitizes Hut78 cells to FasL induced apoptosis. (A) TOX shRNA-transduced and control bulk Hut78 cells were cultured with FasL for 24 hours. Specific apoptosis, which represents the increased apoptosis population after FasL incubation, was analyzed with annexin V binding-based apoptosis assay and compared. Apoptosis profiles of untransduced Hut78 cells, control vector transduced and TOX shRNA transduced Hut78 cells are shown. Numbers denote the percentage of cells within the quadrant. (B) TOX shRNA transduced Hut78 cells revealed increased specific apoptosis in contrast to the control Hut78 cells and parental Hut78 cells. Data in the bar graphs are expressed as the mean±SD of 4 independent transductions and untransduced Hut78 cells. *P<0.05 by Mann-Whitney U test.

FIG. 6 shows down regulation of TOX expression after treatment with the calcineurin inhibitor FK506 in CD4+ T cells. TOX expression levels were evaluated by Western blotting using specific anti-TOX antibody (Sigma Aldrich) 7 days post virus infection. Bulk populations transduced with TOX shRNA showed significant reduction of TOX expression, compared with control populations. FIG. 6 shows three independent transductions. Actin served as an internal reference.

FIG. 7 shows that TOX knockdown reduces the viability of cultured HH cells derived a patient with non-MF/SS CTCL, which is an aggressive form of CTCL. Lentivius-mediated TOX gene transduction results in a growth disadvantage to HH cells. HH-SHC002 (solid line) cells display a growth advantage to HH cells (double solid line) over TOX shRNA cells (TOX knock-down, dotted line). On day 7 or Day 4, the number of viable HH-SHC002 and Hut78-SHC002 cells was significantly higher than the number of viable cells where TOX had been inhibited by TOX shRNAs, respectively. Data were generated from lentivirus transduced bulk cell populations. All experiments were triplicated. P<0.005** by Mann-Whitney U test.

FIG. 8 shows the effect of calcineurin inhibitor treatment on TOX expression in T cells. Peripheral blood mononuclear cells (PBMC) were seeded into 6-well culture plates (2×106/ml) and activated as previously described (Wang et al., 2011). The cells were treated with FK506, a calcineurin inhibitor) at two concentrations: 0.8 ng/ml (L) or 4 ng/ml (H) or PBS (Controls). After initial incubation for 30 minutes, the cells were activated using a cell activation mixture (1% PHA+25 ng/ml PMA), and cultured for 6 more hours. The cells were then harvested and RNA was extracted and used for microarray analysis using Agilent whole genome array (41,000 unique transcripts) as previously described (Wang et al., 2011). After quartile normalization and normalization to GAPDH levels, the individual signal intensity at the TOX gene spots were tabulated and plotted. * p=0.0002; ** p=0.0004

FIG. 9 shows that TOX suppression by lentivirus-mediated shRNA knock down yielded a growth disadvantage to HH CTCL cells (sh), compared with non-TOX-suppressed control HH CTCL cells (SHC002). FIG. 9A. Western blotting confirmed TOX suppression in HH CTCL cells. FIG. 9B. Viability assay showing cell number each day. HH sh cells had a significantly lower number of cells on Days 4/7 and 7/7, compared with control cells. Two tailed t test. ***, P<0.0005. Results were from at least three biological replicates.

FIG. 10 presents data from a colony forming cell (CFC) assay with HH cells from a patient with advanced CTCL. FIG. 10A. Representative image displaying colonies of different sizes in control cells (SHC002) and HH sh cells. FIG. 10B. sh cells generated a smaller number of colonies on Day 11, compared with control HH cells transduced with control vector (SHC002). Paired two-tailed t test, ***, P<0.0005. Results are from three biological replicates.

FIG. 11 shows that TOX suppression leads to increased apoptosis and cell cycle arrest in CTCL (HH) cells transfected with TOX sh to inhibit TOX compared with control CTCL (HH) cells transfected with the control construct (SHC002). FIG. 11A. CTCL cells with TOX knock down (sh) had a higher number of apoptotic cells compared to control cells. FIG. 11B. CTCL cells with TOX knock down (sh) displayed cell cycle arrest (G1 to S, and S to G2), compared with control cells. Two tailed t test. *, P<0.05; **, P<0.005. Results are from at least 3 biological replicates.

### DETAILED DESCRIPTION

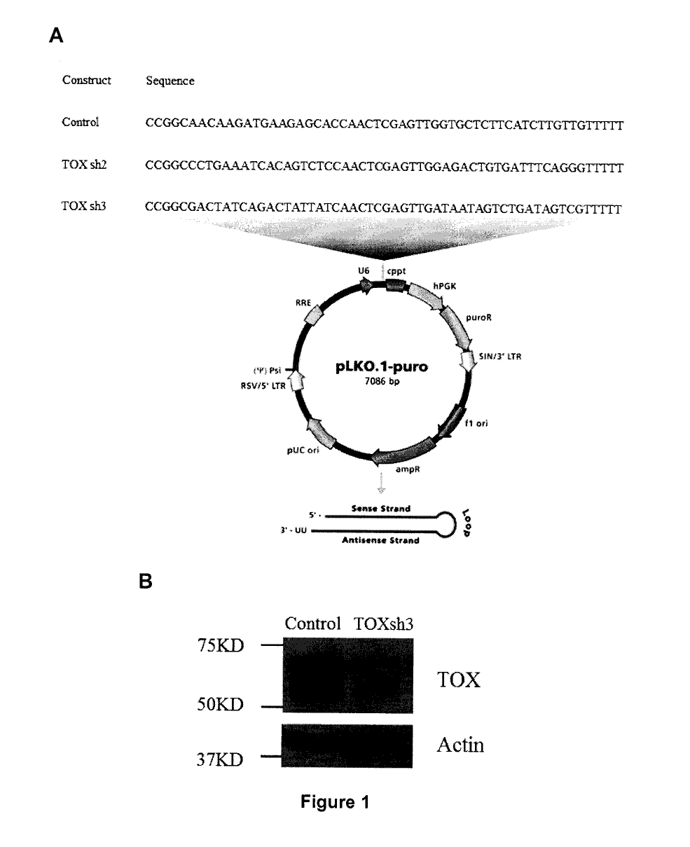

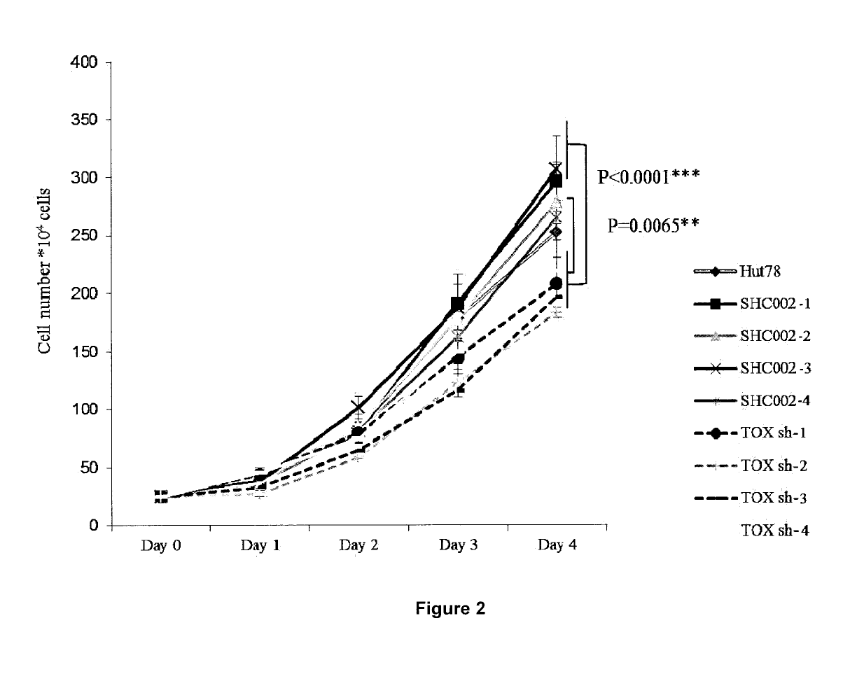

The present inventors have determined that modulating the expression or activity of TOX reduces the proliferation of cancer cells. As shown in FIG. 1, knock-down of TOX using antisense nucleotides results in the reduced proliferation of malignant T cells (Hut78 cells) relative to untransfected Hut78 cells or cells transfected with a control vector. As shown in FIG. 2, inhibiting TOX in Hut78 cells also resulted in reduction in colony size and number relative to normal Hut78 cells and controls in a colony forming assay. Further investigations of Hut78 cells using fluorescence activated cell sorting (FACS) with Hoescht and Pyronin staining demonstrated that inhibiting TOX in Hut78 cells results in G0/G1 and G1/S phase arrest. As shown in Example 2, inhibiting TOX in T cells derived from a patient with non-mycosis fungoides/Sezary syndrome cutaneous T cell lymphoma (MF/SS CTCL) (HH cells) resulted in reduced proliferation and a reduction in colony size and number compared to controls. TOX inhibition in HH cells also resulted in increased apoptosis and cell cycle arrest compared to controls.

Inhibiting TOX therefore represents a highly efficient method of reducing cell proliferation, inducing apoptosis and sensitizing cancer cells to apoptosis of T cell malignancies. The relatively significant effect of TOX inhibition on cell cycle arrest in addition to its observed apoptic effect suggests that therapeutic approaches to cancer treatment that use TOX inhibition may have a favorable safety profile with less cellular toxicity and/or be particular effective in combination with other chemotherapeutic agents.

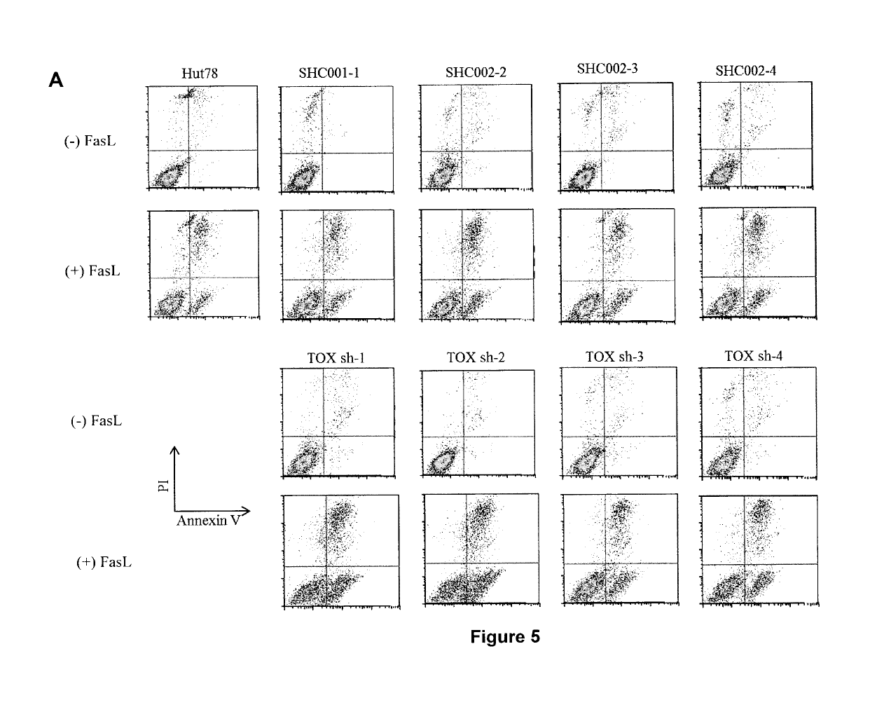

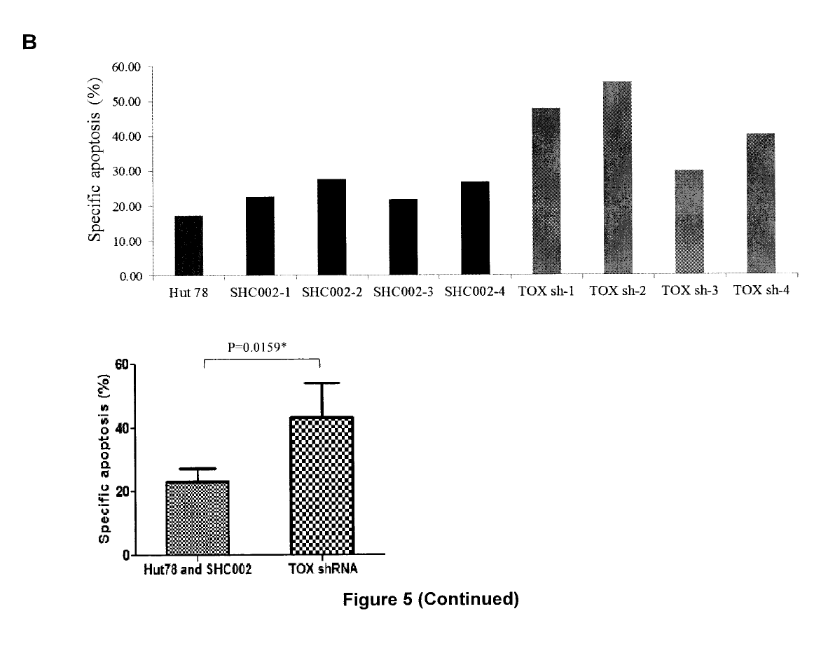

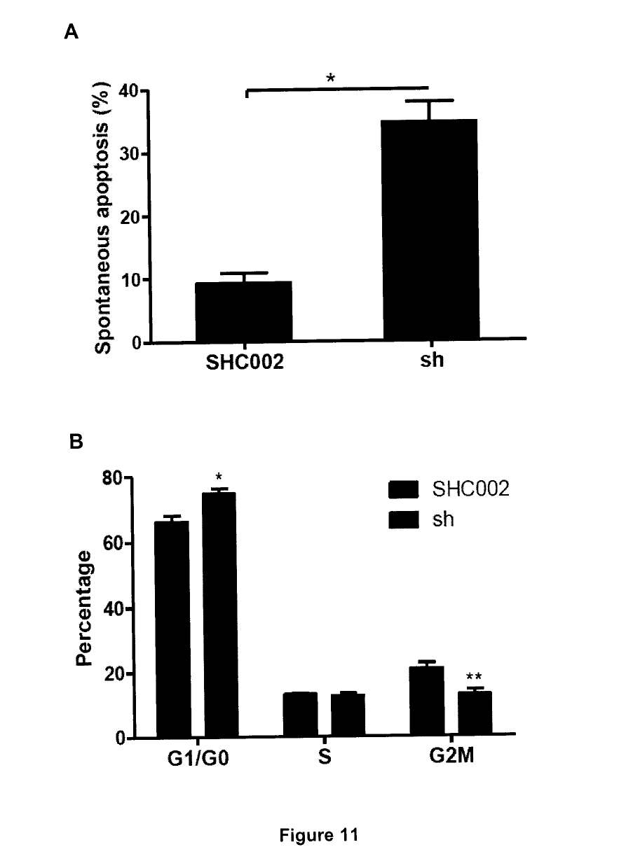

The inventors have also determined that inhibition of TOX in malignant T cells results in a sensitized response to FasL induced apoptosis. As shown in FIG. 5, increased levels of FasL induced apoptosis were observed in Hut78 cells transfected with a Lentivirus encoding a shRNA TOX inhibitor relative to controls. As shown in FIG. 11A TOX inhibition in HH cells resulted in increased levels of spontaneous apoptosis relative to controls. In one aspect, the use of TOX inhibitors is therefore expected to be particularly useful for the treatment of cancer in combination with other chemotherapeutic agents such as cytotoxic agents or antineoplastic agents that target cancer cells other than by phase arrest.

Accordingly, the methods described herein are useful for reducing the proliferation of cancer cells by modulating the expression or activity of TOX. In one embodiment the cancer cells are malignant T cells. Optionally, the cells are in vitro or in vivo. In some aspects, the methods described herein are useful for the treatment of cancer in a subject in need thereof, by modulating the expression or activity of TOX. In some aspects, there is provided the use of a TOX inhibitor for the treatment of cancer. In some embodiment, the cancer is T cell malignancy such as cutaneous T cell lymphoma (CTCL), peripheral T cell lymphoma of T cell leukemia.

As used herein, “TOX” refers to the “Thymocyte selection-associated high mobility group box protein” as well as the gene, nucleic acids and/or polypeptides encoding for TOX. In one embodiment, TOX is encoded by the nucleic acid sequences or polypeptide sequences set forth in database identifiers HGNC: 18988; Entrez Gene: 9760; Ensembl: ENSG00000198846 and UniProtKB: 094900. In one embodiment, TOX refers to the gene, nucleic acids and/or polypeptides as generally described in Wilkinson et al. TOX: an HMG box protein implicated in the regulation of thymocyte selection. Nature Immunology 3 (3): 272-80 (2002), hereby incorporated by reference in its entirety. The coding nucleotides of the mRNA sequence for TOX is set forth in SEQ ID NO: 4.

As used herein “TOX inhibitor” refers to a substance that interferes with the function of TOX. In one embodiment, the TOX inhibitor interferes with the expression or activity of TOX. Examples of TOX inhibitors include, but are not limited to, small organic molecules, antisense nucleic acid molecules and/or substances that bind to and interfere with the TOX protein. In one embodiment, the TOX inhibitor is a small hairpin RNA (shRNA), small interfering RNA (siRNA), morpholino oligonucleotide or aptamer. In one embodiment, the TOX inhibitor is a substance that binds to and interferes with the TOX protein, such as small organic molecule, antibody or antibody fragment. In one embodiment, the TOX inhibitor modulates pathways associated with the expression or activity of TOX. In one embodiment the TOX inhibitor is a calcineurin inhibitor which reduces the expression of TOX. In one embodiment, the calcineurin inhibitor is cyclosporine, FK506 (Tacrolimus), pimerolimus, derivatives thereof or a pharmaceutically acceptable salt thereof. In one embodiment, calcineurin inhibitors may be used in systemic forms or in topical forms to modulate TOX expression to reduce cell proliferation or for the treatment of cancer. Optionally, the TOX inhibitor is conjugated to one or more molecules that improve the safety, delivery or efficacy or the TOX inhibitor. In one embodiment, the TOX inhibitor is conjugated to a cell penetrating peptide.

The term “antisense nucleic acid” as used herein means a nucleotide sequence that is complementary to its target e.g. a TOX transcription product. Optionally, the nucleic acid comprises DNA, RNA or a chemical analog, which binds to the messenger RNA produced by the target gene. Binding of the antisense nucleic acid prevents translation and thereby inhibits or reduces target protein expression. In one embodiment, the antisense nucleic acid binds to all or part of the mRNA for TOX set forth in SEQ ID NO: 4. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules and administering them are known to a person skilled in the art. Examples of shRNA sequences suitable for inhibiting TOX are shown in FIG. 1 of the present application and include TOX sh2 (SEQ ID NO: 2) and TOX sh3 (SEQ ID NO: 3). In one embodiment, the siRNA binds to all or part of the TOX mRNA sequence set forth in SEQ ID NO: 4. In one embodiment, the siRNA binds to all or part of the 5′UTR of the TOX gene.

Aptamers are short strands of nucleic acids that can adopt highly specific 3-dimensional conformations. Aptamers can exhibit high binding affinity and specificity to a target molecule. These properties allow such molecules to specifically inhibit the functional activity of proteins. Thus, in another embodiment, the TOX inhibitor is an aptamer that binds and inhibits TOX.

As used herein, “cancer cells” refer to cells characterized by uncontrolled cell division that are capable of invading adjacent tissues and forming malignant neoplasms. In one embodiment, the cancer cells are malignant T cells. In one embodiment, cancer cells are malignant NK T cells.

As used herein, “T cell malignancy” refers to cancer characterized by the malignant growth of T cells. Examples of T cell malignancy include, but are not limited to, cutaneous T cell lymphoma, peripheral T cell lymphoma and T cell leukemia. As used herein “malignant T cell” refers to a T cell that is undergoing uncontrolled cell division, such as Sezary cells.

As used herein, “cutaneous T cell lymphoma (CTCL)” refers to cancer characterized by lymphoid malignancies derived from T lymphocytes residing in the skin. Subjects with early stage CTCL may present with a rash or skin irritation, which may eventually form plaques and tumors before metastasizing to other parts of the body as the disease progresses. Malignant cells display mature memory T cell markers (i.e. CD4+CD45RO+) but often lose other mature T cell markers such as CD7 and CD26. Subjects with CTCL typically present with the clinical features described above along with the “atypical” histological characteristics of the CTCL cells. These include a slightly larger or angulated nuclear contour, and migration of these cells into the top layer of the skin, the epidermis. In some cases, the cells of CTCL in the peripheral blood carry a unique, but rare multi-lobulated nuclear shape. However, these morphological changes are often difficult to identify, and over lapping cases often occur with benign inflammatory conditions such as chronic dermatitis or allergic reactions to medications. In some cases, it is possible to diagnose CTCL by testing for rearrangement of the T cell receptor gene. However, T cell clonality sometimes occurs in the benign cases, and often CTCL does not present with T cell clonality.

Examples of CTCL include mycosis fungoides and Sezary syndrome. “Mycosis fungoides (MF)” is the most common form of CTCL. Subjects with MF typically have skin manifestations that resemble common benign skin inflammatory conditions such as psoriasis, chronic dermatitis and may present with rash like patches, tumors, or lesions. Malignancies in MF originate from peripheral memory T cells. Optionally, malignant T cells in subjects with MF exhibit a loss of CD7, CD2, CD3 and/or CD28.

“Sezary syndrome” is a leukemic variant of CTCL with systemic involvement. Subjects with Sezary syndrome typically have abnormally shaped lymphocytes, termed Sezary cells, in the peripheral blood. Malignancies in Sezary syndrome originate from central memory T cells. Cancerous cells in Sezary syndrome are typically much larger than in MF and have cerebriform nucleus, and often have loss of CD7.

The term “subject” as used herein refers to any member of the animal kingdom, preferably a human being, including a subject that has, or is suspected of having, cancer. In one embodiment, the subject has, or is suspected of having, a T cell malignancy.

The phrase “reducing cell proliferation” as used herein refers to slowing the rate of proliferation of a cancer cell as compared to the rate of proliferation of the cancer cell in the absence of the substance. For example, in one embodiment contacting a cancer cell with a TOX inhibitor reduces the proliferation of the cell. In one embodiment, “reducing cell proliferation” includes an increase in cell cycle arrest. In one embodiment, “reducing cell proliferation” includes an increase in cell death or apoptosis.

The term “effective amount” as used herein means an amount effective and at dosages and for periods of time necessary to achieve the desired result (e.g. reducing cell proliferation, and/or preventing or treating cancer). For example, an effective amount of a substance may vary according to factors such as the disease state, age, sex, and weight of the subject, and the ability of the substance to elicit a desired response, such as reducing the proliferation of cancer cells or treating cancer, in the subject. In one embodiment, the methods and uses described herein include administering or using an effective active amount of a TOX inhibitor.

As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease or disorder, preventing spread of disease or disorder, delay or slowing of disease or disorder progression, amelioration or palliation of the disease or disorder state, and remission (whether partial or total), whether detectable or undetectable. “Treatment” can also mean prolonging survival as compared to expected survival if not receiving treatment. In one embodiment, the methods and uses described herein are for the treatment of cancer. In one embodiment, the methods and uses described herein are for the treatment of T cell malignancy, optionally cutaneous T cell lymphoma, peripheral T cell lymphoma and/or T cell leukemia.

As used herein, the term “chemotherapeutic agent” refers to a therapeutic agent useful for the treatment of cancer. Examples of chemotherapeutic agents include anti-proliferative or antineoplastic agents that inhibit cell division and/or DNA synthesis. In one embodiment, the chemotherapeutic agent is methotrexate, a retinoid, or histone deacytylase modifiers. In one embodiment, the retinoid is selected from Acitretin (Soriatane®) and bexarotene. In one embodiment the histone deacytylase modifier is selected from vorinostat and romidepsin. In one embodiment, the chemotherapeutic agent reduces the proliferation of malignant T cells.

In one aspect, there is provided a method of reducing the proliferation of cancer cells comprising inhibiting the expression or activity of TOX in the cancer cells. In one embodiment, the method comprises contacting the cells with a TOX inhibitor. In one embodiment, the cancer cells are malignant T cells, such as Cutaneous T-cell Lymphoma (CTCL) cells, peripheral T-cell lymphoma cells or leukemic T cells. Optionally, the cancer cells are in vivo or in vitro.

In another aspect, there is provided a method for treating cancer in a subject in need thereof, comprising inhibiting the expression or activity of TOX. In one embodiment, the method comprises administering to the subject a TOX inhibitor as described herein, such as an antisense nucleic acid molecule, siRNA or other TOX inhibitor. In one embodiment, the TOX inhibitor is a calcineurin inhibitor such as FK506 (Tacrolimus) or a pharmaceutically acceptable salt or thereof. Optionally, the methods for treating cancer described herein include inhibiting the expression or activity of TOX, such as by use of a TOX inhibitor, and the administration or use of a further chemotherapeutic agent, such as an antineoplastic or cytotoxic agent. As shown in Example 1 and FIG. 5, the use of a TOX inhibitor sensitizes malignant T cells to FasL induced apoptosis. Accordingly, the use of a TOX inhibitor in combination with one or more agents that induce apoptosis in cancer cells is expected to be particularly effective for the treatment of cancer. In one embodiment, the methods described herein comprise the co-administration of a TOX inhibitor and a chemotherapeutic agent. In one embodiment, there is provided the use of a composition comprising a TOX inhibitor and a chemotherapeutic agent for the treatment of cancer. In one embodiment, the cancer is a T cell malignancy. Also provided is a composition comprising a TOX inhibitor as described herein and a chemotherapeutic agent, optionally in combination with a pharmaceutically acceptable carrier.

In some embodiments, the methods described herein are useful for the treatment of T cell malignancy. In some embodiments the T cell malignancy is cutaneous T-cell Lymphoma (CTCL), peripheral T cell lymphoma or T cell leukemia. In one embodiment, the cancer is a cutaneous T cell malignancy such as Mycosis Fungoides (MF) or Sezary Syndrome. As shown in Example 1, inhibiting TOX is useful for reducing the proliferation of Hut78 cells. Hut78 is a T cell line derived from a subject with CTCL. A skilled person would therefore consider that inhibiting TOX would reduce the proliferation of other malignant T cells and be useful for the treatment of other cancers such as peripheral T cell lymphoma or T cell leukemia. There are significant similarities between T-ALL and T-cell lymphomas and a common pathogenesis between acute T cell leukemia, acute lymphoblastic leukemia, and CTCL is reflected in the high levels of TOX expression observed in cell lines derived from various T cell malignancies such as Jurkat cells and CCL119 cells.

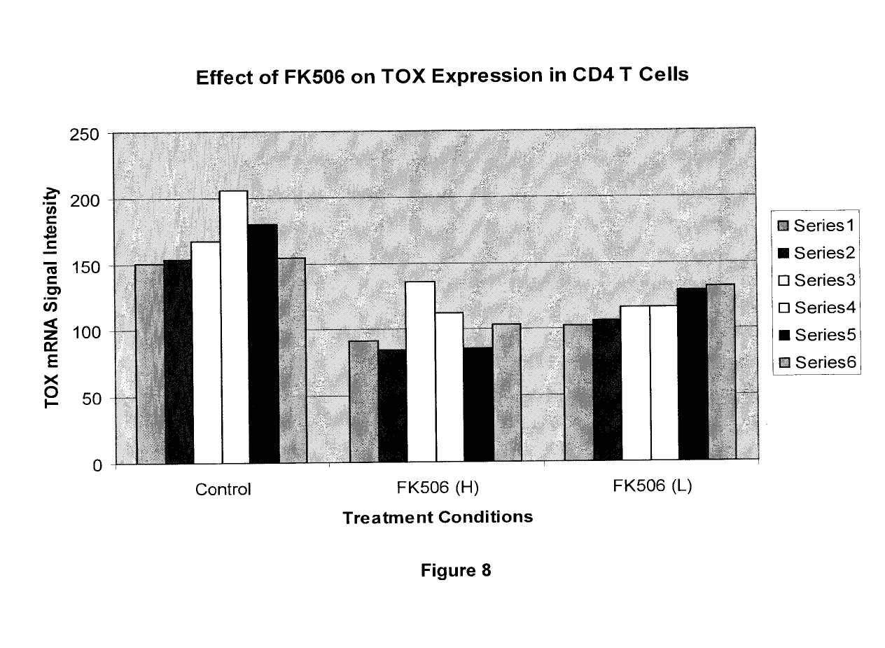

A number of different techniques may be used to inhibit the expression or activity of TOX in order to reduce the proliferation of the cancer cells or for the treatment of cancer as described herein. As shown in FIG. 8, the calcineurin inhibitor FK506 (Tacromlimus) has an inhibitory effect on TOX expression in T cells. Calcineurin inhibitors are therefore expected to reduce the proliferation of cancer cells and be useful in the treatment of cancer, and in particular T cell malignancies. In one embodiment, the methods include the use of a TOX inhibitor that prevents the expression of TOX. In one embodiment, the TOX inhibitor prevents the transcription of TOX mRNA or translation of TOX protein. In one embodiment, the TOX inhibitor is an antisense nucleic acid molecule that binds TOX. In one embodiment, the TOX inhibitor is a nucleic acid that binds to all or part of a nucleic acid encoding for TOX. In one embodiment, the TOX inhibitor is small hairpin RNA (shRNA), small interfering RNA (siRNA) or morpholino oligonucleotides that bind to TOX mRNA. In one embodiment, the TOX inhibitor is a compound that binds to the TOX protein, such as a TOX antibody or aptamer. In one embodiment, the compound is conjugated to a cell penetration peptide that facilitates the cellular uptake of the TOX inhibitor. In one embodiment, the TOX inhibitor is formulated for transdermal delivery or conjugated to a biomolecule to facilitate transdermal delivery. For example, in one embodiment the TOX inhibitor comprises a small interfering RNA, such as the shRNA described herein, conjugated to a synthetic peptide to facilitate transdermal delivery. In one embodiment, such peptides are described in Lin et al. (2012), the contents of which are incorporated by reference in their entirety. In one embodiment the TOX inhibitor is conjugated to a cell permeable or nuclear permeable tag such as a cell penetration peptide. In one embodiment, the TOX inhibitor is conjugated to a molecule that facilitates the transdermal delivery of the TOX inhibitor to the site of CTCL on the skin of a subject. Various such conjugates are known in the art and are included within the embodiments of the present description (see for example, Han et al. 2013, the contents of which are incorporated by reference in their entirety.

In some embodiments, the TOX inhibitors described herein can be prepared by methods known in the art, such as by chemical synthesis of nucleic acid molecules or by using recombinant DNA technologies as known in the art and described in Sambrook et al. (Molecular Cloning: A Laboratory Manual, 3rd Edition, Cold Spring Harbor Laboratory Press, 2001), and other laboratory textbooks. A skilled person will also be able to readily test and identify TOX inhibitors such as by using the cell viability assay or colony forming assay described in Example 1.

In one embodiment, the TOX inhibitors described herein are used in a pharmaceutically acceptable composition which includes an effective quantity of the TOX inhibitor combined with a pharmaceutically acceptable carrier or vehicle. Suitable carriers or vehicles are described, for example, in Remington's Pharmaceutical Sciences (Remington's Pharmaceutical Sciences, 20th ed., Mack Publishing Company, Easton, Pa., USA, 2000). In one embodiment, there is provided a pharmaceutical composition comprising a TOX inhibitor as described herein and one or more chemotherapeutic agents. In one embodiment, the chemotherapeutic agent is a pro-apoptotic agent. In one embodiment, the pharmaceutical composition further comprises a pharmaceutically acceptable carrier or vehicle.

In one embodiment, there is provided a kit comprising a TOX inhibitor as described herein and one or more chemotherapeutic agents. Optionally, the kit includes one or more vials or containers for containing a TOX inhibitor and one or more chemotherapeutic agents. In one embodiment, the kit also includes instructions for the administration or use of the TOX inhibitor and/or chemotherapeutic agents for the treatment of cancer.

The TOX inhibitors can be administered to humans or animals by a variety of methods including, but not restricted to, topical administration, oral administration, aerosol administration, intratracheal instillation, intraperitoneal injection, injection into the cerebrospinal fluid, intravenous injection and subcutaneous injection. Dosages to be administered depend on patient needs, on the desired effect and on the chosen route of administration. Nucleic acid molecules and other substances may be introduced into cells using in vivo delivery vehicles such as liposomes. They may also be introduced into these cells using physical techniques such as microinjection and electroporation or chemical methods such as co-precipitation, pegylation or using liposomes. In some embodiment, the TOX inhibitors are administered to the subject using gene therapy techniques wherein a nucleic acid encoding for a TOX inhibitor is expressed in one or more cells in the subject. In one embodiment, expression of the nucleic acid encoding for a TOX inhibitor is targeted to T cells, optionally malignant T cells.

In one embodiment, there is also provided nucleic acids useful for inhibiting TOX, and associated compositions comprising said nucleic acids. In one embodiment, the shRNA molecules shown in FIG. 1A, or nucleic acid molecules with sequence identity to the shRNA molecules shown in FIG. 1 A are useful for inhibiting TOX.

The following non-limiting examples are illustrative of the present disclosure:

### Examples

### Example 1

### TOX Inhibition Reduces the Proliferation of Malignant T Cells

The inventors investigated the effect of inhibiting TOX on the proliferation of cancer cells. As set out below, inhibition of TOX was determined to reduce the proliferation and increase apoptosis (including FasL induced apoptosis) of cancer cells.

Small hairpin RNAs (shRNA) were developed in order to inhibit the expression of TOX as shown in FIG. 1A. Inhibition of TOX expression by the shRNAs was confirmed by Western blot as shown in FIG. 1B.

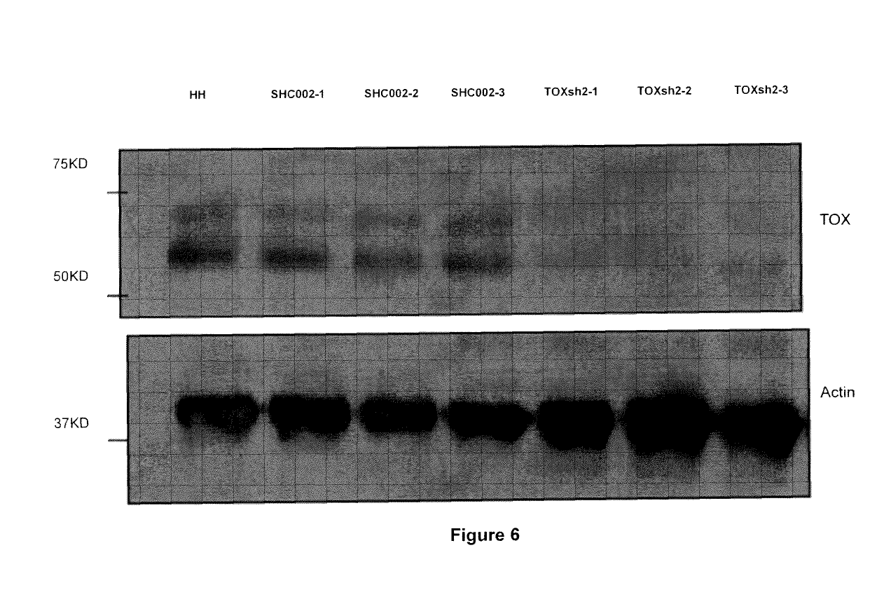

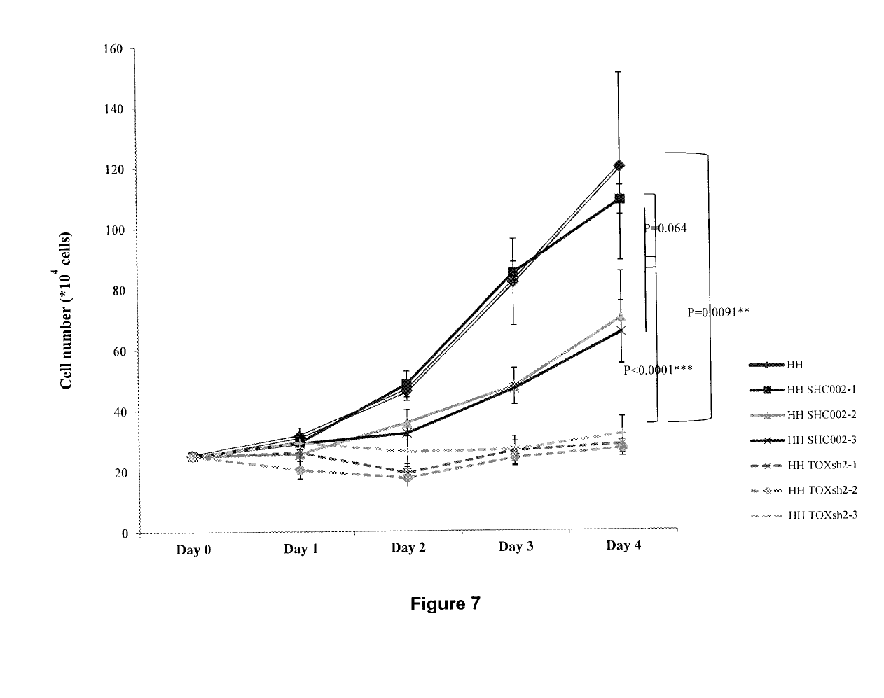

A cell viability assay by trypan blue exclusion method was used to investigate whether inhibiting TOX has an effect on the proliferation of malignant T cells. Hut78 cells from a cell line derived from a subject with CTCL were used as a model for malignant T cells. Lentivirus mediated TOX gene transduction (knock-down) resulted in a growth disadvantage to Hut78 cells relative to untransfected Hut78 cells and cells transducted with a control virus (FIG. 2). A cell viability assay using the HH cell line, which was derived from a patient with aggressive CTCL revealed the same change in the proliferation of cells in response to TOX inhibition (FIGS. 6, 7).

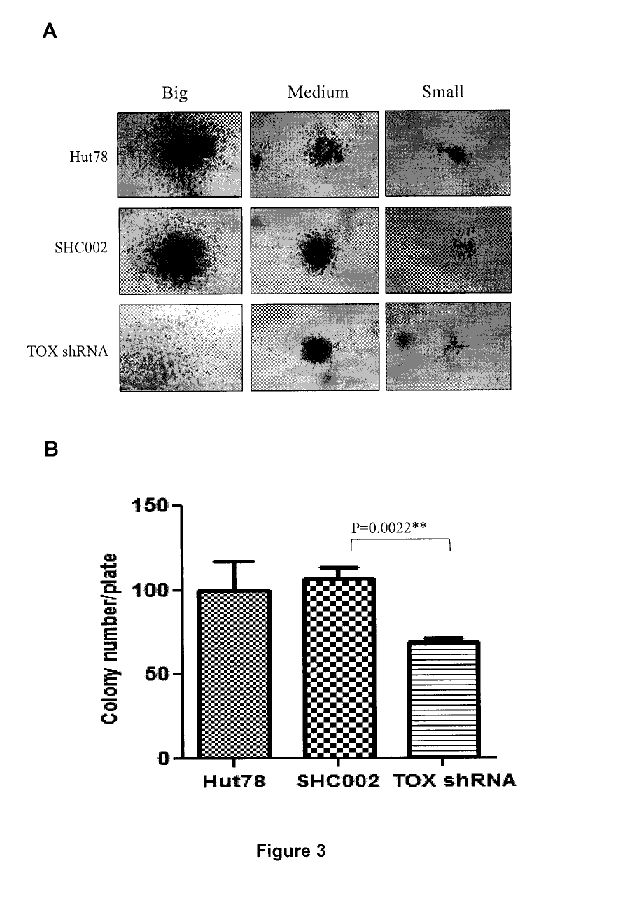

A colony forming cell (CFC) assay was also used to investigate the anti-proliferative effect of inhibiting TOX on cancer cells. Hut78 cells wherein the expression of TOX was inhibited exhibited statistically significant smaller colony size and numbers relative to control cells (FIG. 3).

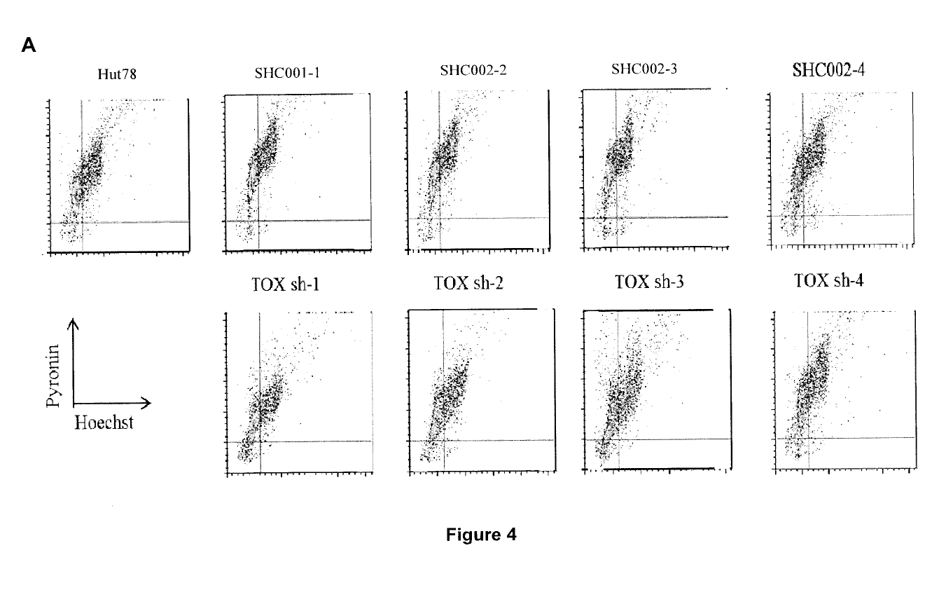

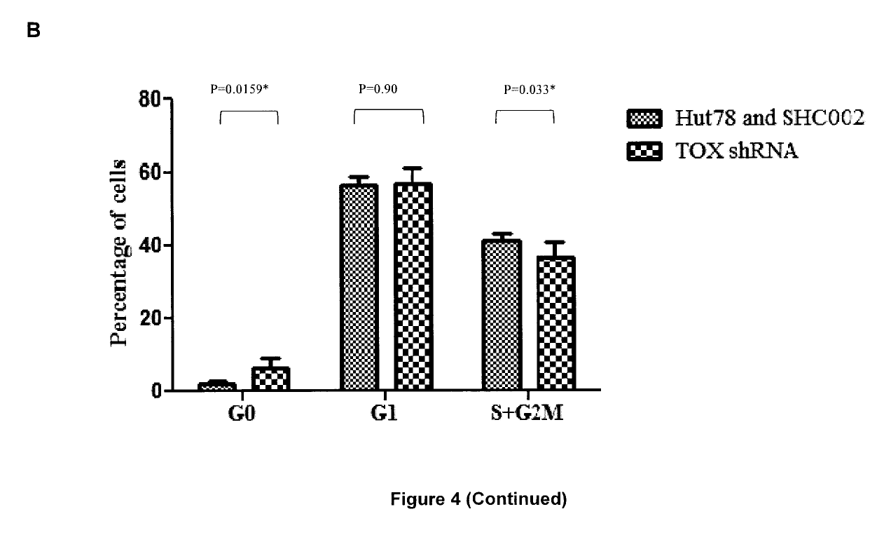

To further investigate the effect of inhibiting TOX expression in Hut78 cells, FACS analysis was used after staining the cells with Hoescht 33342 and Pyronin Y. TOX inhibition resulted in G0/G1 and G1/S phase arrest (FIG. 4).

The effect of TOX inhibition on FasL induced apoptosis was investigated using FACS on Hut78 cells incubated with FasL using an annexin V binding assay. TOX inhibition resulted in an increased sensitivity to FasL induced apoptosis (FIG. 5).

The TOX inhibitor, FK506 (a calcineurin inhibitor) was added to activated T cells in culture and the expression of TOX mRNA was measured by transcriptome analysis. The results showed that TOX mRNA levels were significantly reduced after treatment with FK506 (FIG. 8).

### Example 2

### TOX Inhibition in the Human CTCL Cell Line HH

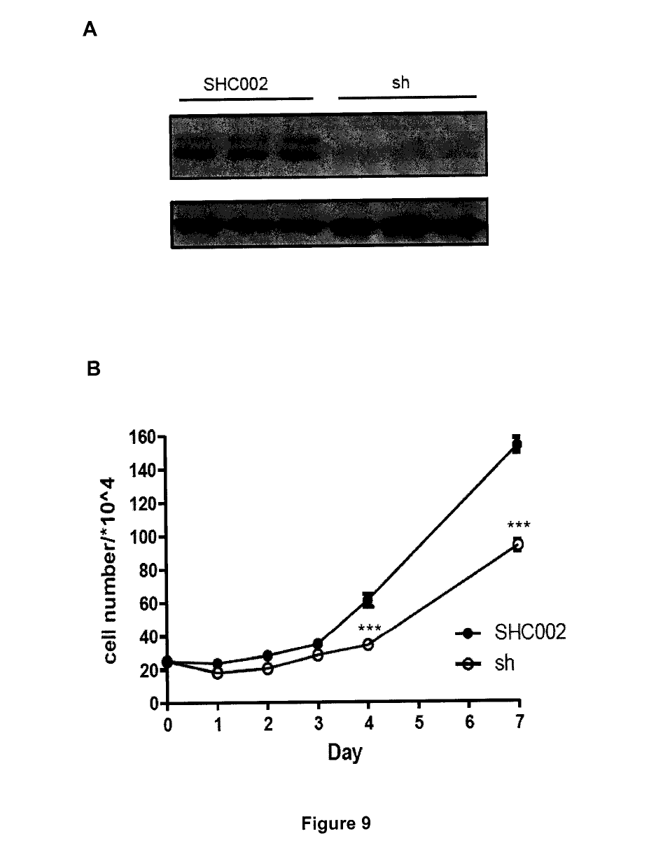

Further investigations of the effects of inhibiting TOX were performed using the HH cell line. The HH cell line is a human cell line derived from a patient with aggressive CTCL and available from the America Type Culture Collection (Rockville Md.). As shown in FIG. 9, TOX suppression in HH cells by lentivirus-mediated sh-RNA resulted in a reduced proliferation of the HH cells relative to control HH cells where TOX was not inhibited.

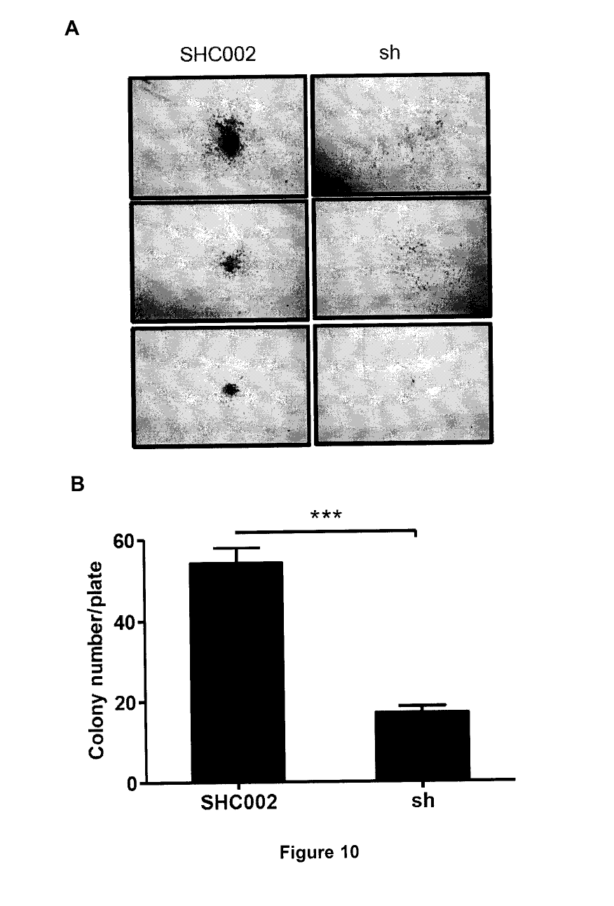

A colony forming cell (CFC) assay was also used to investigate the anti-proliferative effect of inhibiting TOX on HH cancer cells. As shown in FIG. 10, HH cells wherein the expression of TOX was inhibited exhibited statistically significant smaller colony size and numbers relative to control cells where TOX was not inhibited.

As shown in FIG. 11, inhibiting TOX was also observed to increase apoptosis and cell cycle arrest in HH cells where TOX was inhibited using shRNA compared with control HH cells.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents, patent applications and sequence identifiers and/or accession numbers are herein incorporated by reference in their entirety to the same extent as if each individual publication, patent, patent application, sequence identifiers and/or accession numbers was specifically and individually indicated to be incorporated by reference in its entirety.

### REFERENCES

Han et al. “Preventive and therapeutic effects of Smad7 on radiation-induced oral mucositis” Nature Medicine, 2013:19:421-430
Jain et al. “Novel therapeutic agents for cutaneous T-Cell lymphoma” Journal of Hematology & Oncology 2012, 5:24
Lin et al. “A simple, noninvasive and efficient method for transdermal delivery of siRNA” Arch Dermatol Res. 2012 March; 304(2):139-44.
Wang et al. “Deficiency of SATB1 expression in Sezary cells causes apoptosis resistance by regulating FasUCD95L transcription.” Blood 2011 Apr. 7; 117(14):3826-35
Wilkinson, B., J. Y. Chen, et al. (2002). “TOX: an HMG box protein implicated in the regulation of thymocyte selection.” Nat Immunol 3(3): 272-280.

1. A method of reducing the proliferation of one or more cancer cells comprising contacting the cancer cells with a TOX inhibitor.

2.-16. (canceled)

17. A method for treating cancer in a subject in need thereof, comprising modulating the expression or activity of TOX.

18. The method of claim 17, comprising administering to the subject a TOX inhibitor.

19. The method of claim 17, wherein the cancer is T cell malignancy.

20. The method of claim 19, wherein the T cell malignancy is Cutaneous T-cell Lymphoma (CTCL), peripheral T cell lymphoma or T cell leukemia.

21. The method of claim 18, wherein the TOX inhibitor prevents the expression of TOX.

22. The method of claim 21, wherein the TOX inhibitor prevents the transcription of TOX mRNA or translation of TOX protein.

23. The method of claim 18, wherein the TOX inhibitor is a calcineurin inhibitor.

24. The method of claim 23, wherein the calcineurin inhibitor is selected from cyclosporine, FK506 (Tacrolimus), pimerolimus, derivatives thereof and a pharmaceutically acceptable salt thereof.

25. The method of claim 23, wherein the calcineurin inhibitor is FK506 (Tacrolimus).

26. The method of claim 18, wherein the TOX inhibitor is a nucleic acid that binds to a nucleic acid encoding for all or part of TOX.

27. The method of claim 26, wherein the nucleic acid is a small hairpin RNA (shRNA), small interfering RNA (siRNA) or morpholino oligonucleotide.

28. The method of claim 18, wherein the TOX inhibitor binds to the TOX protein, such as a TOX antibody.

29. The method of claim 18, wherein the TOX inhibitor is conjugated to a cell penetrating peptide.

30. The method of claim 29, wherein the cell penetrating peptide is selected from TAT, Angiopep, penetratin, TP, rabies, virus glycoprotein (RVG), prion peptide and SynB.

31. The method of claim 18, wherein the TOX inhibitor is in formulated for transdermal delivery.

32. The method of claim 17, wherein the cancer is Cutaneous T cell malignancy such as Mycosis Fungoides (MF) or Sezary Syndrome.

33. The method of claim 17, further comprising administering to the subject a chemotherapeutic agent.

34. The method of claim 33, wherein the chemotherapeutic agent is selected from methotrexate, retinoids, and a histone deacytylase modifier.

35. A pharmaceutical composition comprising a TOX inhibitor and a chemotherapeutic agent.

36.-39. (canceled)
